(19) United States
(12) Patent Application Publication
(10) Pub. No.: US 2003/0153809 A1
(43) Pub. Date: Aug. 14, 2003

(54) Device for penis enlargement and enhancement

(76) Inventor: Audrey Contente, New York, NY (US)

Correspondence Address: Audrey Contente, Apt. 16C, 211 East 70th Street, New York, NY, 10021, US

(21) Appl. No.: 10/359,110

(22) Filed: Feb. 5, 2003

Related U.S. Application Data
(60) Provisional application No. 60/354,493, filed on Feb. 8, 2002.

(51) Int. Cl. A61F 5/00

(52) U.S. Cl. 600/38

(57) ABSTRACT

The invention relates to a device for providing males with the means to enlarge and enhance the size and shape of their penis without the use of drugs or medicaments, natural or herbal formulas and to further provide males with a means of solving and correcting the problem of a small or misshapen penis and to help the male to overcome the negative psychological and sexual impacts of same, as well as to provide a means to treat female sexual dysfunction (FSD) and female sexual arousal disorder (FSAD) when engaging in sexual contact and sexual intercourse directly as a result of the increased penetration and stimulation from the device which will enhance and increase the ability of females who experience FSD and FSAD as well as all other women to achieve increased sexual arousal, orgasm and total sexual satisfaction.

### FIELD OF THE INVENTION

The invention relates to a device for providing males with the means to enlarge and enhance the size and shape of their penis without the use of drugs or medicaments, natural or herbal formulas and to further provide males with a means of solving and correcting the problem of a small or misshapen penis and to help the male to overcome the negative psychological and sexual impacts of same, as well as to provide a means to treat female sexual dysfunction (FSD) and female sexual arousal disorder (FSAD) when engaging in sexual contact and sexual intercourse directly as a result of the increased penetration and stimulation from the device which will enhance and increase the ability of females who experience FSD and FSAD as well as all other women to achieve increased sexual arousal, orgasm and total sexual satisfaction.

### DESCRIPTION OF RELATED ART

In the United States alone there are about 100 million adult males, many of whom are sexually active, who must deal with women who have female sexual dysfunction (FSD) or female sexual arousal disorder (FSAD) and other women as well who experience the lack of sexual arousal and sexual satisfaction. There are about 100 million adult females in the United States of which over 40 million experience FSD or FSAD. Additionally, there are tens of millions more who, because of their partner's physiological penis size and shape, experience problems with sexual arousal, stimulation and sexual satisfaction and cannot achieve orgasm. Males themselves also experience lack of sexual arousal and sexual satisfaction due to the sexual problems that impact the female population as a result of the physiology regarding penis size and shape.

### SUMMARY OF THE INVENTION

There is a need for a device to provide males with the means to enlarge and enhance the size and shape of their penis while in a semi-erect or an erect state without the use of drugs, medicaments, natural or herbal formulas, chemicals, or any oral or topical formulations or products that can require medical supervision and prescription and without the high cost of the aforementioned, and most importantly, without which any or all of the above mentioned can cause negative side-effects or adverse reactions or substance interactions in order to provide greatly increased sexual arousal, heightened orgasm and total sexual satisfaction for both males and females, whether or not they experience female sexual dysfunction (FSD) or female sexual arousal disorder (FSAD), during sexual contact and sexual intercourse.

There is a need for a device that provides males with a means of solving the problem of a small or misshapen semi-erect or erect penis and helping to overcome the negative psychological and sexual impacts of same.

There is a need for a device that provides a means to treat female sexual dysfunction (FSD) and female sexual arousal disorder (FSAD) when engaging in sexual contact and sexual intercourse.

There is a need for a device that provides a means to treat females who have had physiological changes to their vaginal, cervical and uterine structures, especially as associated with childbearing and hysterectomy, such as internal stretching, when engaging in sexual contact and sexual intercourse.

These and other needs are met by embodiments of the invention, which provide a claim 1.

There are several advantages to the embodiments of the invention.

The invention provides males with the means to enlarge and enhance the size and shape of their penis (while in a semi-erect or an erect state) without the use of drugs, medicaments, natural or herbal formulas, or chemicals. The invention also does not require the use of oral or topical formulations or products that can require medical supervision and prescriptions, which, most importantly, avoids negative side-effects or adverse reactions or substance interactions. Furthermore, cost is low and affordable by the general population while providing for increased sexual arousal, heightened orgasm and total sexual satisfaction for both females, whether or not they experience FSD or FSAD, and males during sexual contact and sexual intercourse.

Moreover, the invention provides women who experience FSD and FSAD a means to increase stimulation for enhancing and increasing the female's ability to self-lubricate which is a major problem associated with these disorders. This ability to self-lubricate will help diminish the physiological and psychological issues associated with painful sexual intercourse and will cause the woman to desire sexual activity and sexual intercourse and thus create a positive and satisfying sexual experience for both the female and the male. Also, the invention provides the female with increased male penile penetration during sexual intercourse; provides increased stimulation, arousal and sensation of the entire vaginal canal, clitoris, ‘G-spot” and cervical area created by the enhanced and enlarged length and girth of the penis; provides increased sexual stimulation, arousal and sensation created by the different protrusion designs on the outside and the inside of the device. The invention leads to total and complete arousal, stimulation, sensation and enhanced and heightened sexual activity and enhanced orgasm for the female, as well as help women achieve orgasms who have been non-orgasmic.

Additional advantages and novel features of the invention will be set forth in part in the description that follows, and in part will become apparent to those skilled in the art upon examination of the following or may be learned by practice of the invention. The advantages of the invention may be realized and attained by means of the instrumentalities and combinations particularly pointed out in the appended claims.

### BRIEF DESCRIPTION OF THE DRAWINGS

Preferred embodiments of the invention will be described with reference to the drawings, which are incorporated in and constitute a part of the specification, in which:

FIGS. 1a-1c are a side view and cross-sectional views of a preferred embodiment of the invention;

FIGS. 2a-2c are a side view and cross-sectional views of another preferred embodiment of the invention;

FIGS. 3a-3c are a side view and cross-sectional views of another preferred embodiment of the invention;

FIGS. 4a-4c are a side view and cross-sectional views of another preferred embodiment of the invention;

FIGS. 5a-5c are a side view and cross-sectional views of another preferred embodiment of the invention;

FIGS. 6a-6c are a side view and cross-section views of another preferred embodiment of the invention;

FIGS. 7a-7c are a side view and cross-sectional views of another preferred embodiment of the invention;

FIGS. 8a-8d are side views and cross-sectional views of the device of another preferred embodiment of the invention;

FIG. 9 is a side view of another embodiment of the invention;

FIG. 10 is a side view of another embodiment of the invention;

FIG. 11 is a side view of another embodiment of the invention;

FIGS. 12a-12d are schematics of several possible alternative configurations of FIG. 9.

### DETAILED DESCRIPTION OF THE PREFERRED EMBODIMENT

These and other objects of the invention are provided for by the inventive size enhancement device for enlarging the length and girth of the penis, which is intrinsically linked to the specific uses and to the specific sexual problems experienced by the male and female as it affects them physiologically, psychologically and emotionally.

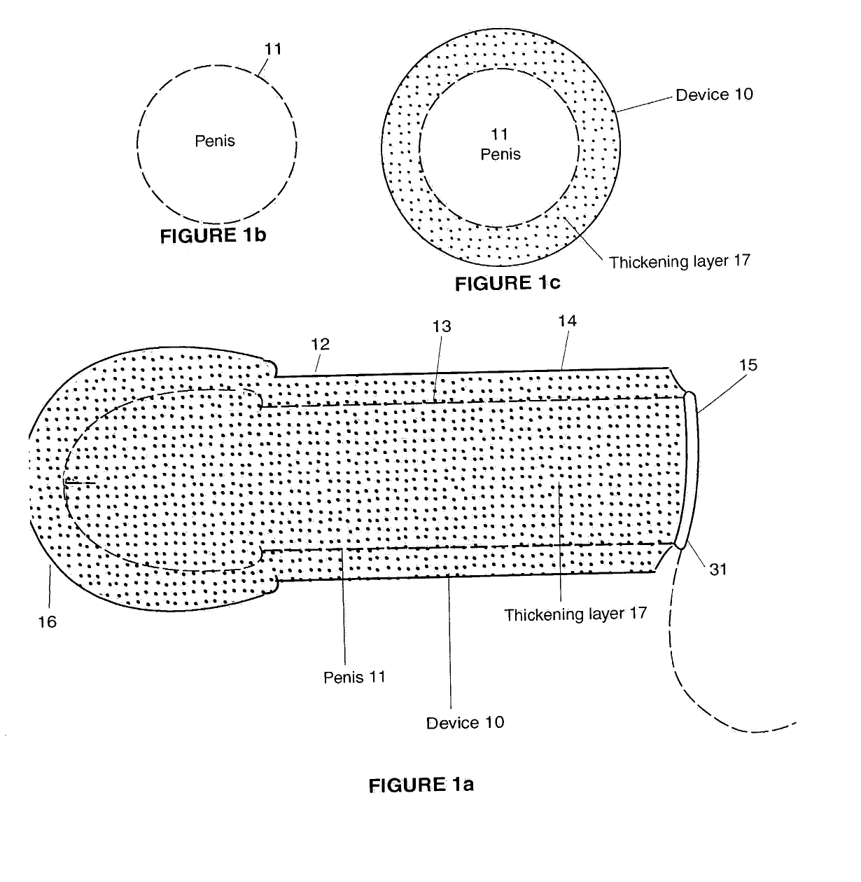

Referring now to the drawings where like numerals refer to like elements and, initially to FIGS. 1a-1c, there will be seen a device 10 for enhancing the size and shape of a penis 11. The device comprises a tubular member 12 that is placed on the shaft and head of a penis. The tubular member 12 has an inner surface 13, an outer surface 14, a first end 15, and a second end 16. In this embodiment, only the first end 15 of the device is open. There is a band 31 disposed at the first end 15 that fits tightly around the base of the shaft of the penis and holds the device in place.

A thickening layer 17 is provided on the inner surface 13 of the device 10 and serves to increase the girth and length of the penis 11. The thickening layer 17 can also add rigidity to the penis. The thickening layer 17 may also provide for a more desirable shape of the penis. Preferably, the tubular member 12 is made from available natural or chemical and porous or non-porous materials such as: thermoplastic elastomers, latex, polyurethane, EVA, animal or vegetable casings or other stretchable or non-stretchable material. Preferably, the thickening layer 17 is made from natural or chemical and porous or non-porous materials such as: gels or foams.

In the first embodiment, the thickening layer 17 is provided over the entirety of the inner surface of the device 10.

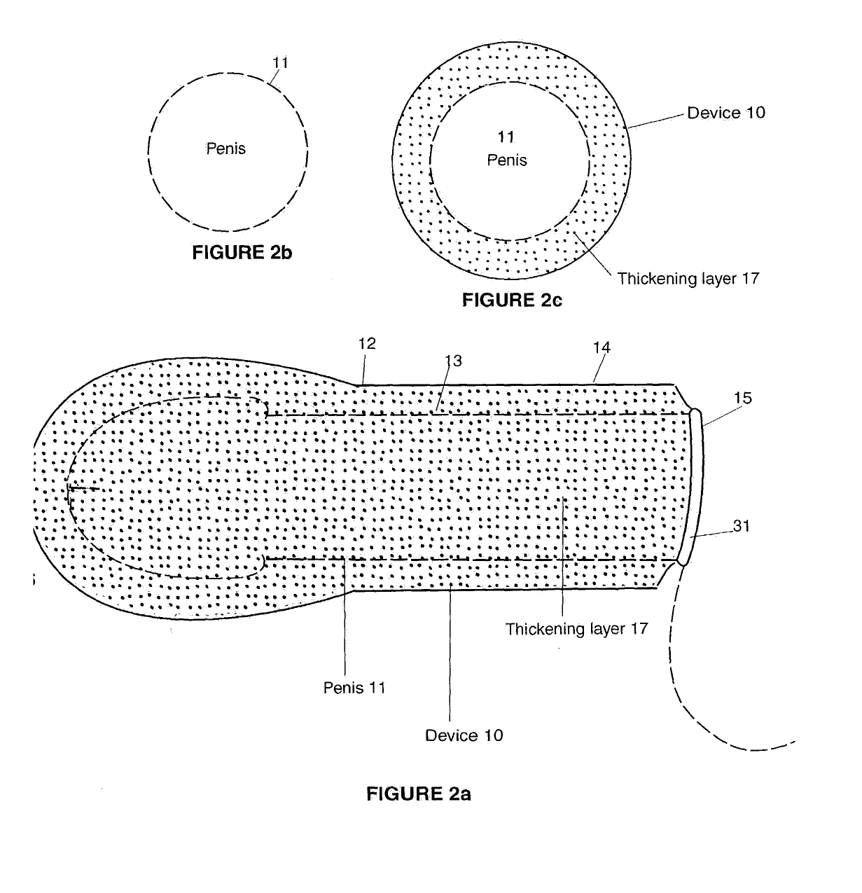

In the second embodiment, which is shown in FIGS. 2a-2c, there is shown a device 10 for enhancing the size and shape of a penis 11 that comprises a tubular member 12 that is placed on the shaft and head of a penis. The tubular member 12 has an inner surface 13, an outer surface 14, a first end 15, and a second end 16. In this embodiment, only the first end 15 of the device is open. There is a band 31 disposed at the first end 15 that fits tightly around the base of the shaft of the penis and holds the device 10 in place.

A thickening layer 17 is provided on the inner surface 13 of the device 10 and serves to increase the size and shape of the penis 11. The thickening layer 17 can also add rigidity to the penis. The thickening layer 17 can also provide for a more desirable shape of the penis. Preferably, the tubular member 12 is made from available natural or chemical and porous or non-porous materials such as: thermoplastic elastomers, latex, polyurethane, EVA, animal or vegetable casing or other stretchable or non-stretchable material. Preferably, the thickening layer 17 is made from natural or chemical, porous or non-porous materials, such as: gels or foams.

In the second embodiment, the thickening layer 17 is provided over the entirety of the inner surface of the device 10.

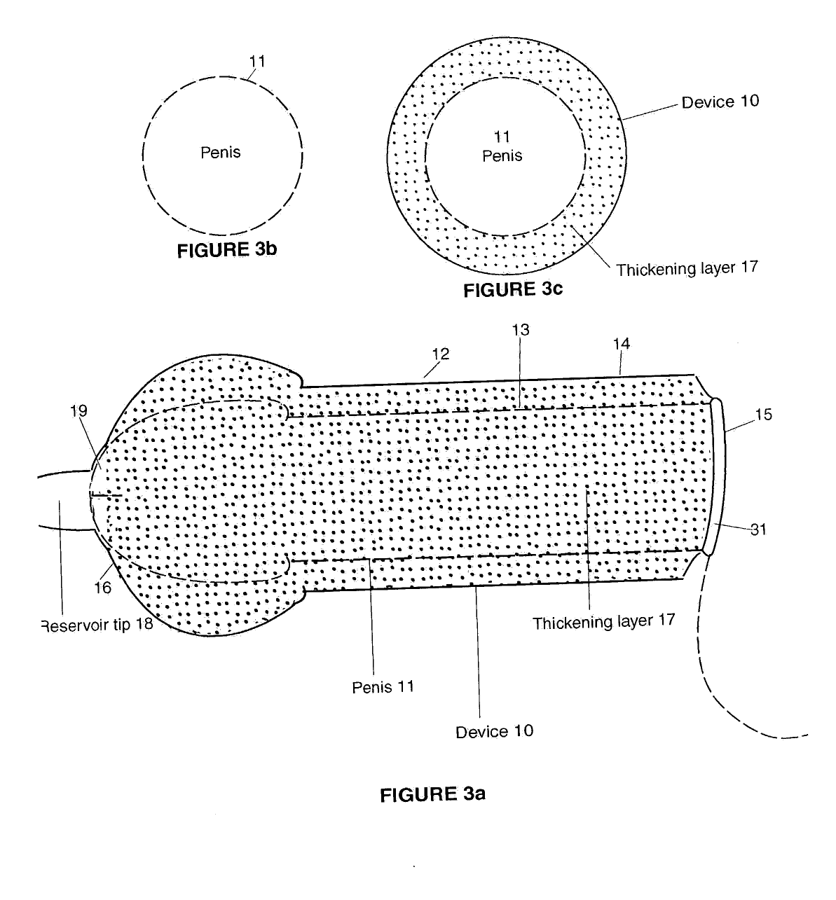

The third embodiment, which is shown in FIGS. 3a-3c, is substantially similar to the first embodiment shown in FIGS. 1a-1c. The third embodiment adds a reservoir tip 18 at the end 16 of the device 10 to contain semen that is ejaculated during intercourse. Accordingly, the thickening layer 17 is not provided on the inner surface in the vicinity of the tip 19 of the penis

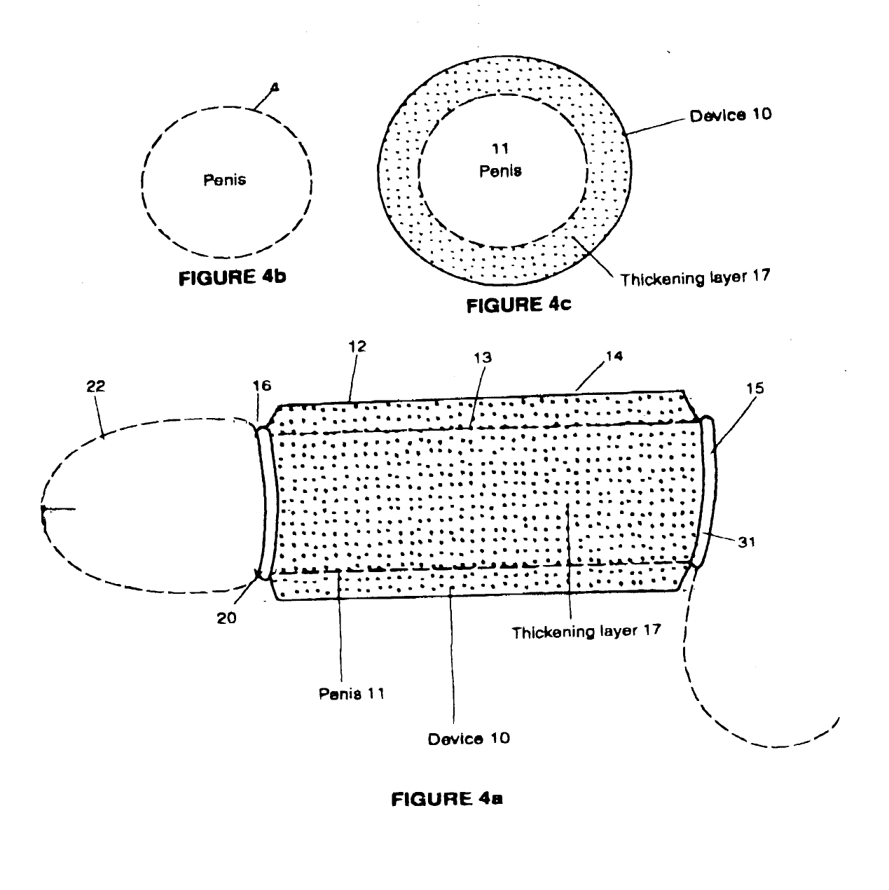

In the fourth embodiment, which is shown in FIGS. 4a-4c, there is shown a device for enhancing the shape of the penis and increasing the girth of a penis that comprises a tubular member 12 that is placed on the shaft of a penis 11. The tubular member 12 has an inner surface 13, an outer surface 14, a first end 15, and a second end 16. In this embodiment, both the first end 15 and the second end 16 are open so that semen ejaculated during intercourse may be transmitted to the female body.

There is a band 31 disposed at the first end 15 that fits tightly around the base of the shaft of the penis 11 that holds the device in place. There is also a band 20 disposed at the second end 16 near the glans 21 of the penis 11 that fits tightly around the upper shaft of the penis 11 that holds the device 10 in place.

A thickening layer 17 is provided on the inner surface 13 of the device 10 and serves to increase the girth of the penis 11. The thickening layer 17 may also add rigidity to the penis 11. The thickening layer 17 can also provide for a more desirable shape of the penis 11. Preferably, the tubular member 12 is made from available natural or chemical and porous or non-porous materials such as: thermoplastic elastomers, latex, polyurethane, EVA, animal or vegetable casing or other stretchable or non-stretchable material. Preferably, the thickening layer 17 is made from natural or chemical and porous or non-porous materials, such as: gels or foams.

In the fourth embodiment, neither the thickening layer 17 nor the device 10 is provided over the entirety of the penis 11.

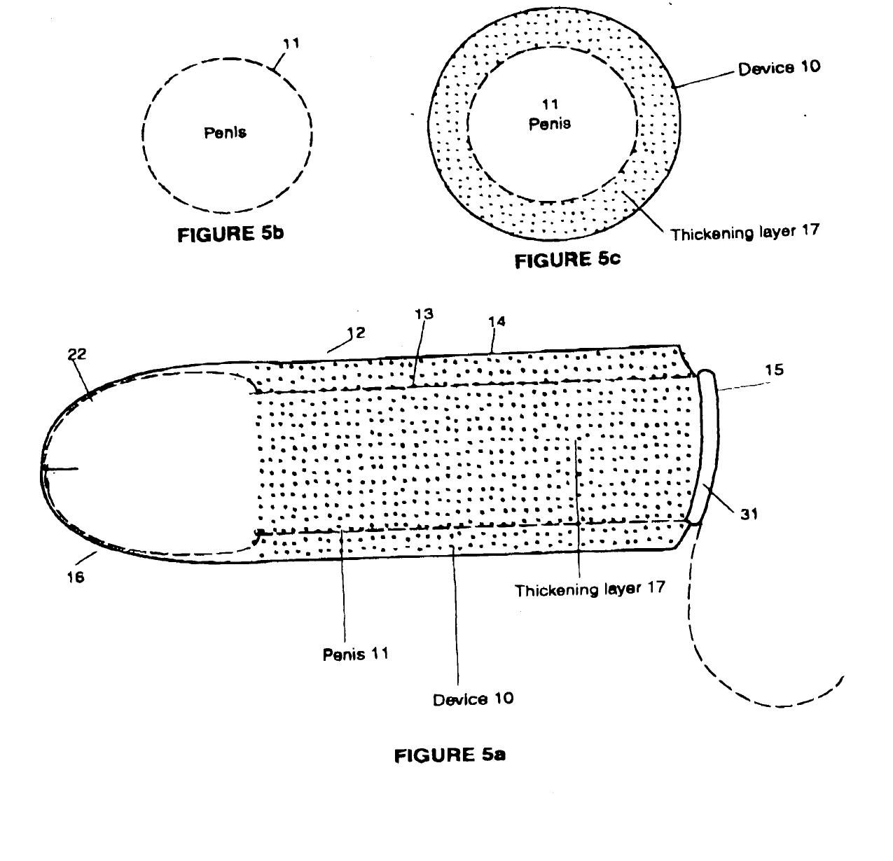

The fifth embodiment is substantially the same as the first embodiment as is seen from a comparison of FIGS. 5a-5c and FIGS. 1a-1c. The main difference is that the thickening layer 17 is not provided over the entirety of the inner surface of the device 10, but is only provided on the portion of the inner surface that is in contact with the shaft of the penis 11. In other words, the thickening layer 17 does not cover the head 22 of the penis 11, which allows for increased sensitivity for the penis 11 during intercourse.

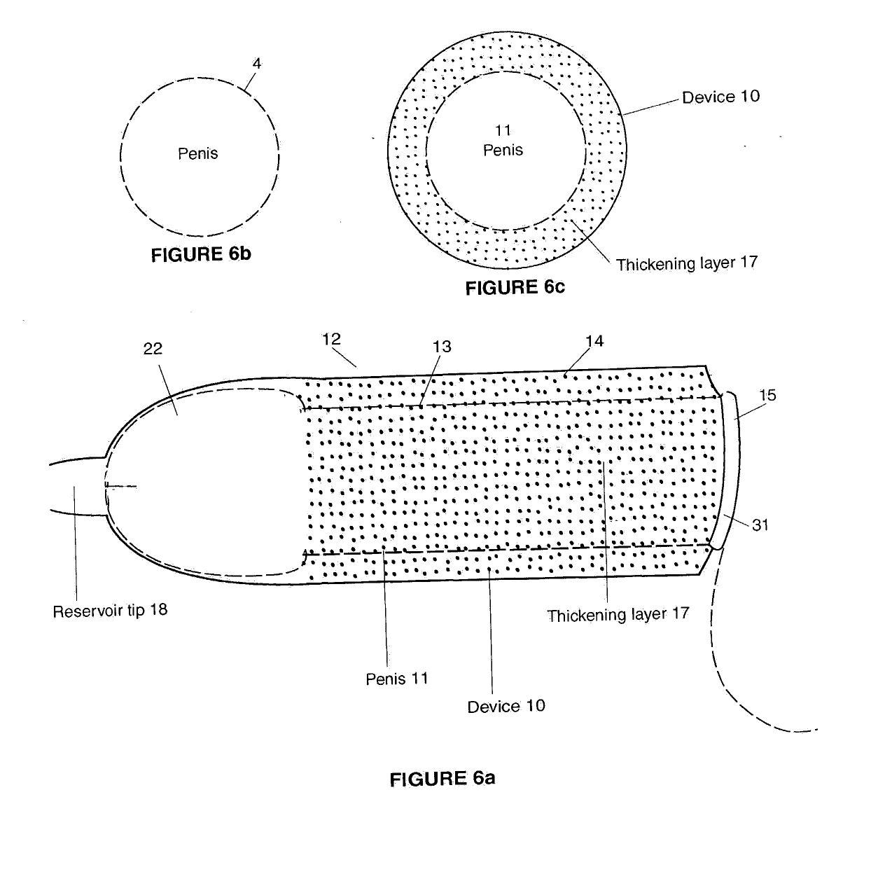

The sixth embodiment, which is shown in FIGS. 6a-6c, is substantially similar to the fifth embodiment shown in FIGS. 5a-5c. The sixth embodiment adds a reservoir tip 18 at the end of the device 10 to contain semen that is ejaculated during intercourse.

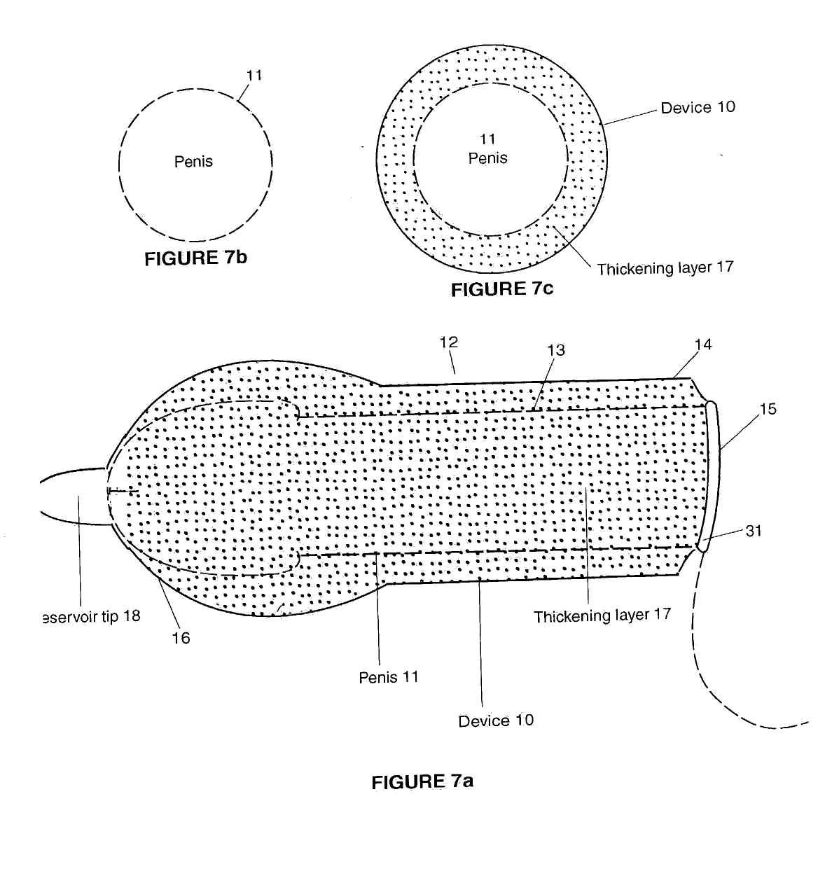

The seventh embodiment, which is shown in FIGS. 7a-7c, is substantially similar to the second embodiment shown in FIGS. 2a-2c. The seventh embodiment adds a reservoir tip 18 at the end of the device 10 of the second embodiment to contain semen that is ejaculated during intercourse. Accordingly, the thickening layer 17 is not provided on the inner surface in the vicinity of the tip of the penis 11.

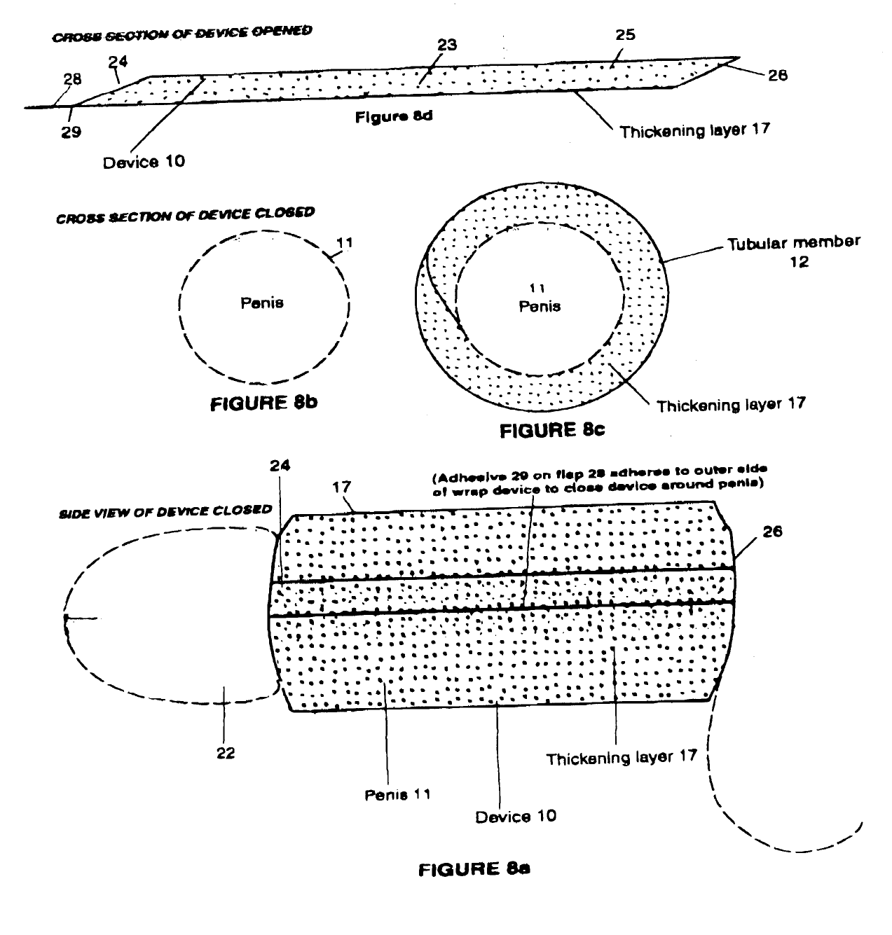

In the eighth embodiment, which is shown in FIGS. 8a-8d, there is shown a device 10 for enhancing the size of a penis 11 that comprises initially a flat member. The flat member has an inner surface 23, an outer surface 25, a first end 24, and a second end 26. The flat member also has a flap 28 that extends from one lengthwise edge of the outer surface 25. The flap contains an adhesive 29 of any bio-compatible material to secure the device in position.

With this embodiment, the flat member is wrapped around the penis 11, such that there is no overlap of the thickening layer 17, that is, the thickening layer abuts itself at the lengthwise sides of the device 10 as shown in FIG. 8c. This forms a tubular member 12 that is substantially similar to the embodiment of FIG. 4. The adhesive 29 is placed over the outer surface of the other lengthwise flap 28 to hold the device 10 as a tubular member 12.

The tubular member 12 formed by this embodiment has both ends open so that semen ejaculated during intercourse may be transmitted to the female body.

A thickening layer 17 is provided on the inner surface of the device 10 and serves to increase the girth of the penis 11. The thickening layer 17 can also add rigidity to the penis 11. The thickening layer 17 can also provide for a more desirable shape of the penis 11. Preferably, the tubular member 12 is made from available natural or chemical and porous or non-porous materials such as: thermoplastic elastomers, latex, polyurethane, EVA, animal or vegetable casing or other stretchable or non-stretchable material. Preferably, the thickening layer 17 is made from natural or chemical and porous or non-porous material such as: gels or foams.

In the eighth embodiment, the thickening layer 17 is provided over the entirety of the inner surface of the device 10.

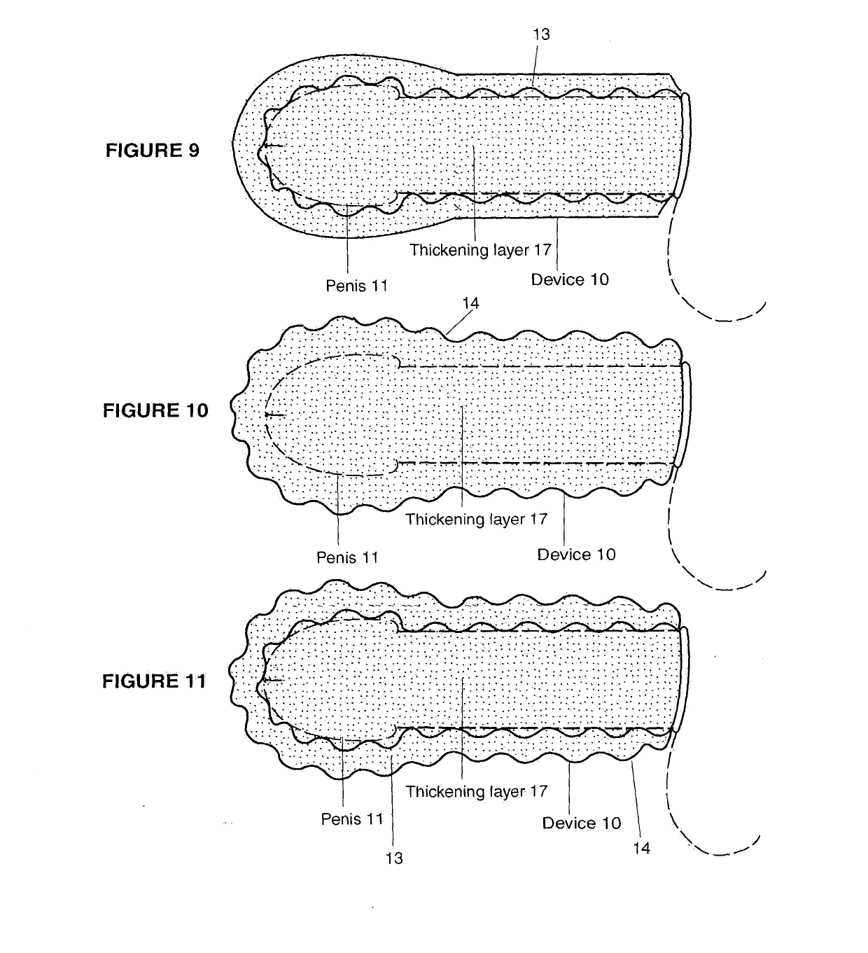

Each of the eight embodiments has been shown with the thickening layers with smooth surfaces. FIGS. 9-11 show alternative arrangements of thickening layers having contoured surfaces. In FIG. 9, only the inner surface 13 is contoured. In FIG. 10, only the outer surface 14 is contoured. In FIG. 11, both the inner surface 13 and the outer surface 14 are contoured.

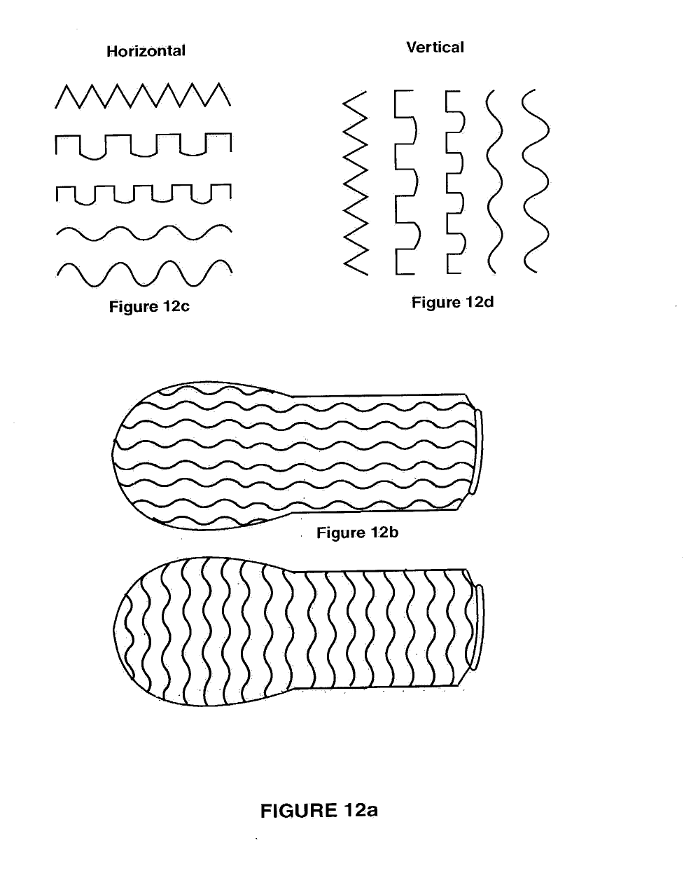

FIGS. 12a-12d provide more detail regarding the contours of the thickening layers. For example, as shown in FIG. 12b, the contours can run along the length of the device. As shown in FIG. 12a, the contours can run around the circumference of the device. FIGS. 12c and 12d provide a variety of differing contemplated contour shapes of the thickening layer, but more contours are envisioned

The embodiments of the invention provide the male with: (a) increased penis length to allow for increased penile penetration during sexual intercourse; (b) increased stimulation of the penis created by the increase in penile girth against the walls of the vaginal canal; (c) increased stimulation and arousal created by the different protrusion designs of the device on the penis; (d) additional heat created by the thickening layer, inside the device, against the entire penis which increases sexual arousal, sensation and stimulation; and, all of which can lead to total and complete sexual arousal, stimulation, sensation and heightened sexual activity and enhanced orgasm for the male.

This, and other embodiments of the invention, provide a means to increase stimulation to women who experience FSD and FSAD in order to improve, enhance and increase the female's ability to self-lubricate because these women often cannot self-lubricate. This ability to self-lubricate will help diminish the physiological and psychological issues associated with painful sexual intercourse and will cause the woman to desire sexual activity and sexual intercourse and thus create a positive and satisfying sexual experience for both the female and the male. The embodiments of the invention can also provide all females, whether or not they experience FSD or FSAD, with: (a) increased male penile penetration during sexual intercourse; (b) increased stimulation, arousal and sensation of the genitals to include the entire vaginal area, clitoris, ‘G-spot’ and cervical area created by the enhanced and enlarged length and girth of the penis; (c) increased sexual stimulation, arousal and sensation created by the different protrusion designs on the outside of the device; and, all of which can lead to total and complete arousal, stimulation, sensation and enhanced and heightened sexual activity and enhanced orgasm for the female, as well as help those women achieve orgasms who have been non-orgasmic.

What is claimed is:

1. A device for enhancing the size and shape of a penis, comprising: a tubular member adapted for placement on the shaft of the penis and having an inner surface, an outer surface, a first end, and a second end, at least one of the first and second ends is open; and, a thickening layer provided on the inner surface.

2. A device as claimed in claim 1, further comprising a band at the first end that secures the first end to the base of the penis.

3. A device as claimed in claim 1, further comprising a reservoir tip at the second end of the tubular member.

4. A device as claimed in claim 1, wherein both the first and the second ends are open.

5. A device as claimed in claim 1, further comprising a band at the second end that secures the second end to the penis.

6. A device as claimed in claim 1, further comprising an adhesive flap that secures the tubular member to the penis.

7. A device as claimed in claim 1, wherein the outer surface is smooth.

8. A device as claimed in claim 1, wherein the outer surface is contoured.

9. A device as claimed in claim 1, wherein the inner surface is smooth.

10. A device as claimed in claim 1, wherein the inner surface is contoured.
